(19) United States
(12) Patent Application Publication
(10) Pub. No.: US 2008/0039776 A1
(43) Pub. Date: Feb. 14, 2008

(54) DEVICES FOR ENDOSCOPIC THERAPEUTIC CELL DELIVERY

(76) Inventors: Ragae Ghabrial, Cincinnati, OH (US); Mark S. Ortiz, Milford, OH (US); William B. Fox, New Richmond, OH (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/463,016

(22) Filed: Aug. 8, 2006

(51) Int. Cl. A61M 5/00; A61F 2/06

(52) U.S. Cl. 604/59; 604/57; 623/1.41

(57) ABSTRACT

A medical device having an elongate flexible tube extending between a proximal end and a a distal end. The device has a lumen extending between the proximal and distal ends. The lumen contains therapeutic cells and is sized to receive a plunger. The plunger is movable through at least a portion of the lumen. The device further includes an actuator at the proximal end of the elongate flexible tube, and a delivery member at the distal end of the elongate flexible tube.

### FIELD OF THE INVENTION

The present invention relates broadly to methods and devices for the minimally invasive transplantation of therapeutic cells. The present invention has application in conventional surgical instrumentation as well application in robotic-assisted surgery.

### BACKGROUND OF THE INVENTION

Loss of organ function can result from congenital defects, injury or disease. Many times treatment with drugs or surgery is not in itself sufficient. One approach for treatment has been to transplant donor organs or tissue into the patient. Nevertheless, such procedures are still associated with a high degree of morbidity and mortality, in addition to being very expensive and requiring extensive perioperative and post-operative care. While organ transplantation has proliferated, the most significant factor restricting the applicability of whole organ transplantation operations, however, is the limited availability of donor organs.

Certain disease states involving organ failure can be successfully treated by replacing only a small proportion of the organ mass with populations of donor cells. Such donor cells (referred to herein as “therapeutic cells”) are obtained from a donor organ by mechanical disruption or enzymatic digestion of the parenchyma of a donor organ. Some therapeutic cells may be in the form of single cells and others may be in the form of clusters of cells where each cluster comprises more than one cell. Advances in cell culture techniques have made it possible to maintain donor cells such as hepatocytes in a viable and functional state in vitro for extended periods of time until they are transplanted into a recipient. Successful transfer of such cellular transplants into animal recipients has recently been demonstrated for both liver cells (See Rhim et al., Science 263, 1149 (1994)) and heart muscle cells (See Soonpaa et al., Science 264, 98 (1994)).

To optimize the process of transplantation of therapeutic cells, the transplant procedure should be performed in a non-invasive manner without sacrificing the accuracy required for successful delivery of the therapeutic cells. Endoscopic surgical instruments have been adopted for performing minimally invasive surgical procedures via the use of a patient's natural orifice. A commercially available endoscope in its current state may not be capable of delivering a small volume of fluid comprising millions of therapeutic cells that are sensitive to pressure, temperature and pH fluctuations.

Placement of the therapeutic cells into the correct anatomic location may be important if the therapeutic cells are to function properly after implantation. The correct placement may require stabilizing the receiving site and accurate maneuvering of the delivering device to such a site. Visualization of the placement site may be of great importance as well.

### SUMMARY OF THE INVENTION

In accordance with the present invention, there is provided a medical device having an elongate flexible tube extending between a proximal end and a a distal end. The device has a lumen extending between the proximal and distal ends. The lumen contains therapeutic cells and is sized to receive a plunger. The plunger is movable through at least a portion of the lumen. The device further includes an actuator at the proximal end of the elongate flexible tube, and a delivery member at the distal end of the elongate flexible tube.

### BRIEF DESCRIPTION OF THE DRAWINGS

The invention will be more fully understood from the following detailed description taken in conjunction with the accompanying drawings, in which:

FIG. 1 is a perspective view of one embodiment of a device of the current invention, showing all the main component of the device including an actuator and a delivery member connected by an elongate flexible tube covered by an outer protective sheath;

FIG. 2 is a longitudinal cross-sectional view taken across the delivery member showing the plunger and the elongate flexible shaft adhered thereto. It also shows the outer protective sheath at a proximal position with the needle's distal tip in an uncovered position;

FIG. 3 is a perspective view of the distal portion of the device shown in FIG. 1 showing the delivery member, the needle, and also a camera all housed by the outer protective sheath;

FIG. 4 is a perspective view of the proximal portion of the device shown in FIG. 1 showing the actuator including the plunger actuator, articulation knobs and several fittings for a light source, a camera and a vacuum source; and

FIG. 5 is a side view of a central region of the device in FIG. 1 uncovering the articulation cables and a camera cable extending along side the elongate flexible tube.

### DETAILED DESCIPTION OF THE INVENTION

Certain exemplary embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting exemplary embodiments and that the scope of the present invention is defined solely by the claims appended hereto. The features illustrated or described in connection with one exemplary embodiment may be combined with the features of other embodiments. Such modifications and variations are intended to be included within the scope of the present invention.

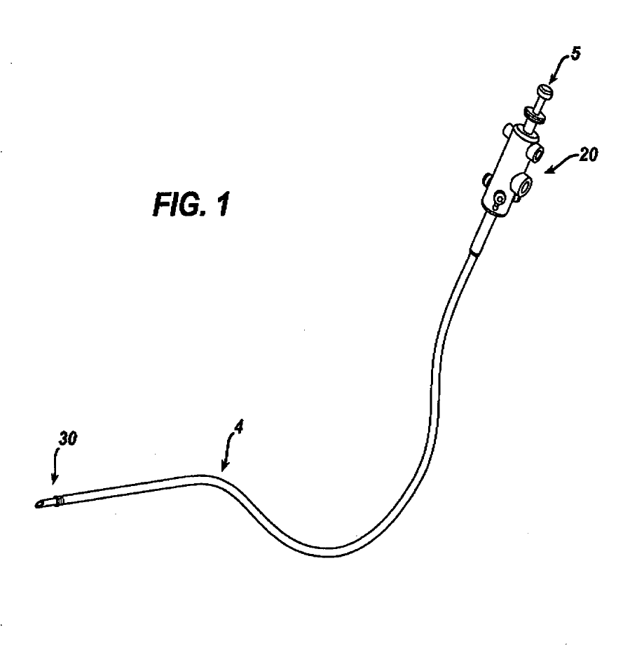

FIG. 1 depicts a medical device of the current invention. The medical device may be comprised of an elongate flexible tube (1) fitted with a delivery member (30) at its distal end and an actuator (20) at its proximal end. The elongate flexible tube (1) may have enough length and elasticity to be inserted into a patient's natural orifice and maneuvered in a patient's lumen towards a target site of the patient. The elongate flexible tube (1) or a portion of which may act as a reservoir for holding therapeutic cells to be delivered to the target site. The inner diameter of the elongate flexible tube (1) may be varied to provide a space suitable for carrying therapeutic cells to be delivered. The elongate flexible tube (1) may have any inner diameter in the range from 1 to 20 mm. The inner surface of the elongate flexible tube (1) may be sterilized to maintain a sterile environment suitable for housing therapeutic cells.

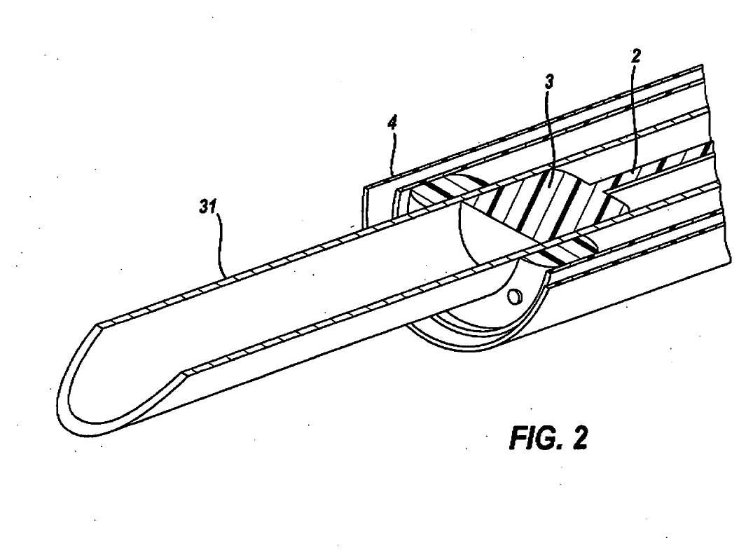

The elongate flexible tube (1) may also house a plunger (3) that is movable between retracted and fully inserted positions within the elongate flexible tube (1) to intake or discharge therapeutic cells through the delivery member (30). A longitudinal cross-section view of the medical device, as shown in FIG. 2, shows the plunger (3) housed inside the elongate flexible tube (1). The plunger (3) may comprise an outer covering of a preferably elastic material that provides a dynamic seal through continuous pressure against the inner walls of elongate flexible tube (1).

The plunger (3) may be fitted with an elongate flexible shaft (2) at its proximal end (FIG. 2) that extends proximally through the elongate flexible tube (1) and through a central lumen within the actuator (20) of the device. The geometry, diameter, and material composition of the elongate flexible shaft (2) may be varied to enhance flexibility while still maintaining enough rigidity to transfer forces to the plunger (3) as is known and understood in the art. The elongate flexible shaft (2) may have any diameter in the range of 0.1 to 19.5 mm. In one embodiment, the diameter of the elongate flexible shaft (2) may be slightly less than that of the elongate flexible tube (1), possibly with a lubricating material minimizing friction between the outer surface of the elongate flexible shaft (2) and the inner walls of the elongate flexible tube (1) upon movement of the elongate flexible shaft (2) through it. This configuration may minimize buckling of the elongate flexible shaft (2) while transferring forces to the plunger (3) leading to a more efficient force transfer. The flexible elongate shaft may be fitted with a plunger actuator (5) at its proximal end (FIG. 1) such that applying axial forces to the plunger actuator (5) at the actuator (20) is translated into axial movement of the plunger (3).

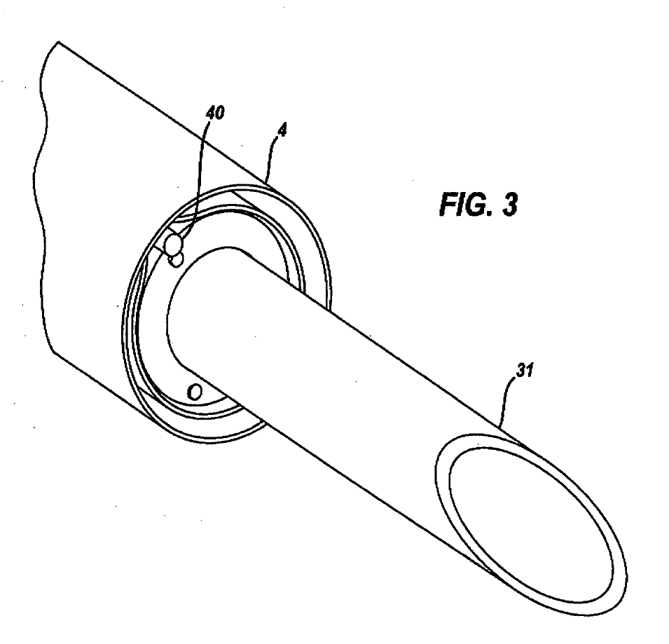

FIG. 3 shows the delivery member (30) of the medical device comprising a needle (31) at its distal end. The inner lumen of the needle (31) and the inner lumen defined by the elongate flexible tube (1) communicate with each other to define a continuous lumen. In one embodiment the inner diameter of the elongate flexible tube (1) and that of the needle (31) may be substantially the same, as shown in FIG. 2, allowing the plunger (3) to slide through the needle (31) ejecting any remaining therapeutic cells that may reside in the needle (31) following regular injection. Those with ordinary skill in the art will appreciate the importance of minimizing any loss of the therapeutic cells in the needle (31) achieved by allowing the plunger (3) to slide through the needle (31). The inner diameter for the needle (31) may be varied depending on the size of the therapeutic cells being delivered to minimize friction against the needle's inner walls, which may be a stress inducing factor on the therapeutic cells. Other stress inducing factors that may influence the survival of the therapeutic cells during the delivery process include changes in pH and in temperature. The medical device of the current invention may be equipped with sensors to continuously monitor the therapeutic cells while housed for delivery into the appropriate target site. A pH sensor and a temperature sensor may provide valuable real time feedback to the operator on the status of the therapeutic cells.

The delivery member (30) may also comprise an outer sheath (4) (FIG. 3) disposed over at least a portion of the elongate flexible tube (1) and movable relative to the elongate flexible tube (1) between a distal position and a proximal position where at its distal position, the outer protective sheath completely covers the distal tip of needle (31). The outer sheath (4) may also be configured to allow the distal tip of needle (31) to penetrate tissue only when the protective sheath is advanced into the tissue to be penetrated. The outer sheath (4) may also be equipped with a sensor to determine the distance the distal tip of the needle (31) travels through the tissue to ensure accuracy of the delivery and prevent over puncturing.

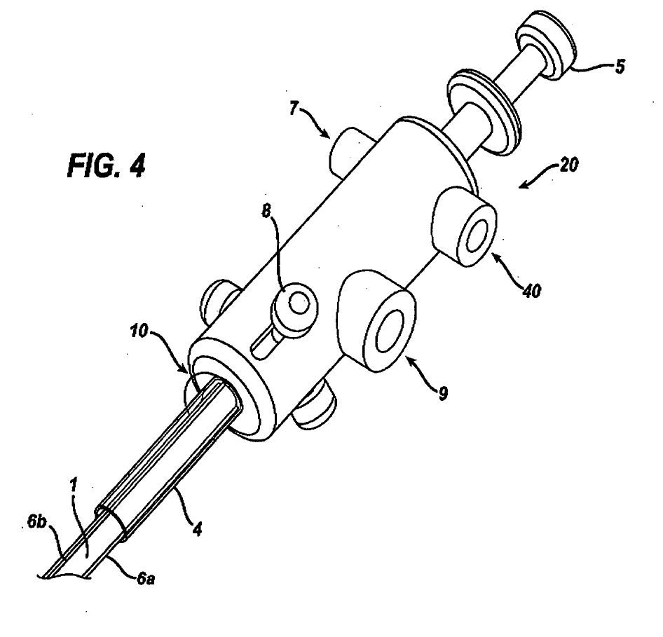

FIG. 4 shows the actuator (20) of the medical device of the current invention. The actuator (20) may comprise a suction fitting (7) to which a vacuum source may be connected to apply suction through the outer sheath (4). In one embodiment the actuator (20) may comprise an outer sheath actuation knob (10) for axially sliding the outer sheath (4) over the elongate flexible tube (1). Further more, the outer sheath actuation knob (10) maybe configured that upon rotating it may open and close a valve controlling the suction flow path through the outer sheath (4). The actuator (20) may also comprise a light source fitting (9) to which a fiber optic cable may be connected to allow light from a fiber optic source to be transmitted through the entire length of the medical device and emitted at the distal end to illuminate the internal body cavity where the delivery of the therapeutic cells is being performed. A continuous view of the operation site may be transmitted to a video monitor via a camera (40) at the distal portion of the medical device. The camera (40) may be connected to a camera cable (41), which may be extended along side the elongate flexible tube (1) and coupled to a camera fitting (42) at the actuator (20) to which the video monitor's cable may be attached. The actuator (20) may also comprise articulation knobs for articulating a distal portion of the elongate flexible tube (1). Each articulation knob may be connected to an articulation cable such that the movement of the articulation knob may result in tensioning or releasing the cable, which in turns results in bending or flexing the elongate flexible tube (1). In one embodiment, movement of the articulation knobs may be an axial sliding movement. In another embodiment, it could be a rotary movement. The articulation knobs can also optionally include a locking mechanism associated therewith and configured to maintain the cables in a fixed position in order to keep the elongate flexible tube (1) in a desired articulated or angular orientation. While the locking mechanism can have a variety of configurations, in one exemplary embodiment the locking mechanism can be in the form of a clamp that is effective to clamp down onto the cables and thereby prevent movement of the cables to lock the working end in a desired orientation.

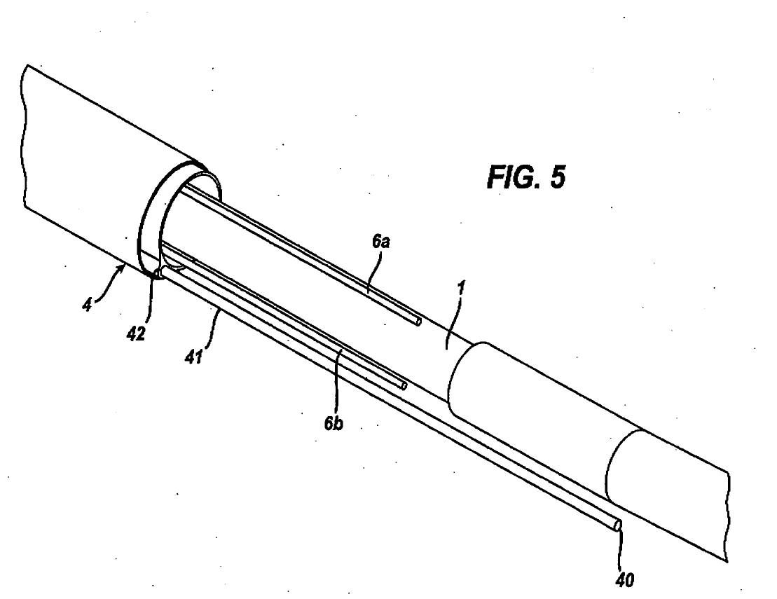

Referring now to FIG. 5, the medical device of the current invention may also include a plurality of articulation cables (6a & 6b & 6c) extending along a length of the elongate flexible tube (1) and equally spaced apart from one another around its outer circumference. The articulation cables may be configured to slide relative to an axis of the elongate flexible tube (1) and to apply tension to the elongate flexible tube (1) to cause at least a portion of which to flex and bend. In one embodiment, one or more of the articulation cables could be in the form of coil pipes. In yet another embodiment, one or more of the articulation cables could be in the form of flexible rods that allow for transfer of compressive forces to the elongate flexible tube (1) causing at least a portion of which to bend or flex. The number and location of the articulation cables can vary. For example, four articulation cables may be spaced approximately 90° apart from one another around the circumference of the elongate flexible tube (1). In yet another example, three articulation cables (6a & 6b & 6c) can be spaced approximately 120° apart from one another around the circumference of the elongate flexible tube (1). FIG. 5 shows such embodiment with only two of the three articulation cables (6a & 6b) in view and the third articulation cable (6c) being hidden behind the elongate flexible tube (1). Each articulation cable may be extended through a separate pathway, such as a lumen, formed on or around the elongate flexible tube (1). In another embodiment, all articulation cables may be housed in one common lumen as illustrated in FIG. 5 showing a view that exposes articulation cables (6a & 6b).

In other embodiments, the articulation cables disclosed herein used to effect articulation of the elongate flexible tube (1) can be formed from an electroactive polymer material. Electroactive polymers (EAPs), also referred to as artificial muscles, are materials that exhibit piezoelectric, pyroelectric, or electrostrictive properties in response to electrical or mechanical fields. In particular, EAPs are a set of conductive-doped polymers that change shape when an electrical voltage is applied. The conductive polymer can be paired to some form of ionic fluid or gel and electrodes, and the flow of ions from the fluid/gel into or out of the conductive polymer can induce a shape change of the polymer. Typically, a voltage potential in the range of about 1V to 4 kV can be applied depending on the particular polymer and ionic fluid or gel used. It is important to note that EAPs do not change volume when energized; rather they merely expand in one direction and contract in a transverse direction. Thus, EAP articulation cables can replace the articulation cables previously disclosed herein, and the actuator (20) can be configured to activate an energy source to selectively deliver energy to one or more of the articulation cables.

People with ordinary skill in the art will appreciate that the device of the current invention with all or some of its components may be utilized alone or in association with an endoscope. The endoscope may be entered through a patient's natural orifice and extended until it reaches the desired target site. The medical device may then be advanced through the endoscope's accessory channel or exterior to the endoscope along its axial length through an external accessory channel. Once in position, the articulation knobs can be rotated to articulate the elongate flexible tube (1) and thereby position the delivery member (30) to be facing the target site. In particular, a first articulation knob may be rotated in a first direction to apply tension on one of the articulation cables bending the elongate flexible tube (1) in its direction to establish contact between the target site and the distal end of the outer sheath (4). At this point, Suction may be activated to stabilize the target site. The needle (31) may then be advanced into target site while a sensor monitors its advancement to prevent over puncturing. Once the needle (31) is in place, the plunger actuator (5) may transmit forces to the plunger (3) through the elongate flexible shaft (2) leading to ejection of the therapeutic cells through the needle's distal tip into the target site. At this point, the medical device may be retracted to reload with another volume of the therapeutic medium to be injected into another target site.

In another embodiment, the medical device may be loaded with more than one volume of therapeutic cells to be delivered to several target sites. A sensor on the plunger actuator (5) may provide feedback to the operator of the complete delivery of a desired volume of therapeutic cells by monitoring movement of the plunger actuator (5) to a pre-identified first position, which corresponds to delivery of a first volume of therapeutic medium. The needle (31) may then be retracted to a proximal position where the needle's distal tip is completely covered by the outer sheath (4) and the medical device is articulated to a second target site where the delivery process is repeated. The advantage of this embodiment is elimination of the reloading step, which may be time consuming. In the case where the medical device is used alone independent of an endoscope, it may be fitted with means for penetrating a body wall such as a stomach wall to enter into a natural body cavity such as a peritoneal cavity in order to deposit therapeutic cells in between tissue layers of a certain organ within that cavity. Such means can include for example graspers or curved wires to retract the body wall and a needle knife or other mechanical means for puncturing through the body wall to provide access into the body cavity.

In other aspects, the current invention also presents a method for treating a patient. The method of the current invention may involve delivering therapeutic cells to a patient's internal viscera by entering a natural orifice of the patient and navigating through the patient's alimentary tract lumen to a portion of the alimentary tract wall. The method may further involve puncturing the alimentary tract wall into a patient's internal cavity, locating a target site within the internal cavity, and delivering therapeutic cells into target site. In one embodiment, the target site may be a patient's heart muscle, the natural orifice may be a patient's mouth, the alimentary tract lumen may be the esophagus and the internal cavity may be the thoracic cavity.

In yet another embodiment, the various devices disclosed herein, including portions thereof, can be designed to be disposed of after a single use, or they can be designed to be used multiple times. In either case, the device can be reconditioned for reuse after at least one use. Reconditioning can include any combination of the steps of disassembly of the device, followed by cleaning or replacement of particular pieces, and subsequent reassembly. By way of example, the medical device shown in FIG. 1 can be reconditioned after the device has been used in a medical procedure. The device can be disassembled, and any number of the particular pieces can be selectively replaced or removed in any combination. For example, for the plunger (3), flexible elongate shaft and/or the plunger actuator (5) may be separated from the actuator (20), which in turn may be separated from the elongate flexible tube (1). Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A medical device, comprising:
a. an elongate flexible tube having a proximal end, a distal end, and a lumen extending between the proximal and distal ends;
b. the lumen containing therapeutic cells and sized to receive a plunger, wherein the plunger is movable through at least a portion of the lumen;
c. an actuator at the proximal end of the elongate flexible tube; and
d. a delivery member at the distal end of the elongate flexible tube.

2. The medical device of claim 1, wherein the device further comprises an outer sheath slideably movable over at least a portion of the delivery member

3. The medical device of claim 2, wherein the outer sheath is connected to a vacuum source.

4. The medical device of claim 1, wherein the device further comprises one or more elongate flexible members extending axially relative to the elongate flexible tube and having a distal portion of which anchored relative to the elongate flexible tube.

5. The medical device of claim 4, wherein the device further comprises one or more pipes surrounding the one or more elongate flexible members.

6. The medical device of claim 1, wherein the delivery member comprises a hollow needle with a sharp distal end capable of passing a fluid carrying therapeutic cells.

7. The needle of claim 6, wherein the hollow needle is also a light source for illumination.

8. The medical device of claim 1, wherein the device further comprises sensors for monitoring the temperature and pH of a fluid comprising therapeutic cells.

9. The medical device of claim 1, wherein the plunger further comprises an outer covering that provides a dynamic seal through continuous pressure against the inner wall of elongate flexible tube.

10. The medical device of claim 1, wherein the device further comprises a camera.

11. A medical device, comprising:
a. an elongate flexible tube having a proximal end, a distal end, and a lumen extending between the proximal and distal ends, the flexible tube is sized and shaped to fit through a working channel of an endoscope;
b. the lumen containing therapeutic cells and sized to receive a plunger, wherein the plunger is movable through at least a portion of the lumen;
c. an actuator at the proximal end of the elongate flexible tube; and
d. a delivery member at the distal end of the elongate flexible tube.

12. The medical device of claim 11, wherein the device further comprises an outer sheath slideably movable over at least a portion of the delivery member

13. The medical device of claim 12, wherein the outer sheath is connected to a vacuum source.

14. The medical device of claim 11, wherein the device further comprises one or more elongate flexible members extending axially relative to the elongate flexible tube and having a distal portion of which anchored relative to the elongate flexible tube.

15. The medical device of claim 14, wherein the device further comprises one or more pipes surrounding the one or more elongate flexible members.

16. The medical device of claim 11, wherein the delivery member comprises a hollow needle with a sharp distal end capable of passing a fluid carrying therapeutic cells.

17. The needle of claim 16, wherein the hollow needle is also a light source for illumination.

18. The medical device of claim 11, wherein the device further comprises sensors for monitoring the temperature and pH of a fluid comprising therapeutic cells.

19. The medical device of claim 11, wherein the plunger further comprises an outer covering that provides a dynamic seal through continuous pressure against the inner wall of elongate flexible tube.

20. A medical device, comprising:
a. An endoscope, an elongate flexible tube having a proximal end, a distal end, and a lumen extending between the proximal and distal ends, the flexible tube is sized and shaped to fit through a working channel of the endoscope;
b. the lumen containing therapeutic cells and sized to receive a plunger, wherein the plunger is movable through at least a portion of the lumen;
c. an actuator at the proximal end of the elongate flexible tube; and
d. a delivery member at the distal end of the elongate flexible tube.
